(19) United States
(12) Patent Application Publication
(10) Pub. No.: US 2005/0049424 A1
(43) Pub. Date: Mar. 3, 2005

(54) 6-Hydroxy isoflavones derivatives and medicaments involving same

(76) Inventors: Graham Edmund Kelly, New South Wales (AU); Andrew Heaton, New South Wales (AU)

Correspondence Address: FINNEGAN, HENDERSON, FARABOW, GARRETT & DUNNER;LLP, 1300 I STREET, NW, WASHINGTON, DC, 20005, US

(21) Appl. No.: 10/493,390

(22) PCT Filed: Oct. 25, 2002
(86) PCT No.: PCT/AU02/01442

(30) Foreign Application Priority Data
Oct. 25, 2001 (AU) PR 8464

(51) Int. Cl. C07D 307/77; C07D 323/02

(52) U.S. Cl. 549/234; 549/434

(57) ABSTRACT

Isoflavone compounds of the formula (I) or (II) Where R can be R [symbol] 7? or OR [symbol] 7? or O and the formula (III) is either a single or double bond extends to Y then Y is an optionally substituted benzyl. W, R [symbol] 1?, Z [symbol] o? R [symbol] 2? and R [symbol] 7? are as defined in the specification. The compounds are useful for the treatment of certain diseases and disorders, including cancer and inflammation.

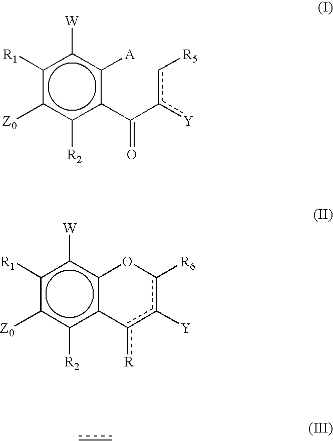

### FIELD OF THE INVENTION

This invention relates to compounds, formulations, drinks, foodstuffs, methods and therapeutic uses involving, containing, comprising, including and/or for preparing certain isoflavone compounds and analogues thereof. In particular, the invention relates to 6-hydroxy substituted isoflavones, derivatives thereof and medicaments involving same.

### BACKGROUND OF THE INVENTION

Naturally-occurring plant isoflavones are known to possess a wide range of fundamental biological effects on human cells including anti-oxidation and the up-regulation and down-regulation of a wide variety of enzymes and signal transduction mechanisms. Mitotic arrest and cytotoxicity of human cancer cells, increased capillary permeability, increased cellular adhesion, increased response of vascular smooth muscle cells to vaso-relaxants, and agonism of estrogen receptors, are just a few examples of the responses of animal cells to the biological effects of naturally-occurring isoflavonoids.

A range of therapeutic benefits as a result of these biological outcomes have been identified including the treatment and prevention of pre-menopausal symptoms such as pre-menstrual syndrome, endometriosis, uterine fibroids, hyperlipidaemia, cardiovascular disease, menopausal symptoms such as osteoporosis and senile dementia, alcoholism, benign prostatic hypertrophy, and cancers such as prostate, breast and large bowel carcinomas [see WO 93/23069; WO 96/10341; U.S. Pat. No. 5,424,331; JP 62-106017; JP 62-106016; U.S. Pat. No. 5,516,528; JP 62-106016A2; JP 62-106017A2; JP 61-246124; WO 98/50026; WO 99/43335; WO 00/49009; WO 00/644,438; WO 99/48496].

While over 700 different naturally occurring isoflavones are described, only a few are confirmed as having potential therapeutic benefits in animals including humans. These include daidzein, genistein, formononetin, biochanin and glycitein. These and all naturally occurring isoflavones are found in nature as the monomeric form either in a free state, or, more likely, bound to a carbohydrate moiety (glycoside). The isoflavone has to be separated from this moiety before it becomes biologically active.

A number of compounds with a structure related to naturally occurring plant isoflavones are also described as having biological properties with potential therapeutic benefit to animals including humans. These include compounds that are naturally occurring metabolites of plant isoflavones produced by bacterial fermentation by gut flora and embrace compounds such as equol and 0-desmethylangolensin [WO 93/23069; WO 98/08503; WO 01/17986; WO 00/66576]. Also included in this group is the synthetic isoflavonoid ipriflavone, which is developed for the treatment of postmenopausal osteoporosis [WO 91/14429] and a wide range of synthetic isoflavonoid analogues [WO 98/08503].

Despite the considerable research and accumulated knowledge in relation to isoflavonoid compounds and derivatives thereof, the full ambit of therapeutically useful isoflavonoid compounds and their activities is yet to be realised. Moreover, there is a continual need for new, improved or at least alternative active agents for the treatment, prophylaxis, amelioration, defence against and/or prevention of various diseases and disorders.

A requirement accordingly exists for new generation compounds that exhibit important pharmacological effects for use as prophylactics and in therapy.

### SUMMARY OF THE INVENTION

According to an aspect of this invention there is provided isoflavone compounds and analogues thereof of the general formula (I): 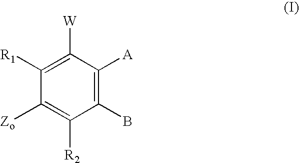
in which
R₁ and R₂ are independently hydrogen, hydroxy, OR₉, OC(O)R₁₀, OS(O)R₁₀, CHO, C(O)R₁₀, COOH, CO₂R₁₀, CONR₃R₄, alkyl, haloalkyl, aryl, arylalkyl, thio, alkylthio, amino, alkylamino, dialkylamino, nitro or halo, and
Z_{O} is hydroxy, or
R₂ is as previously defined, and R₁ and Z_{O} taken together with the carbon atoms to which they are attached form a five-membered ring selected from 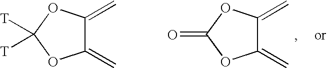
R₁ is as previously defined, and R₂ and Z_{O} taken together with the carbon atoms to which they are attached form a five-membered ring selected from 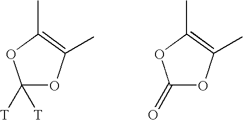
   and
W is R₁, A is hydrogen, hydroxy, NR₃R₄ or thio, and B is selected from 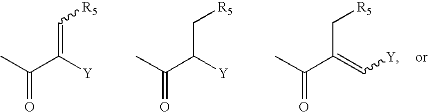
W is R₁, and A and B taken together with the carbon atoms to which they are attached form a six-membered ring selected from 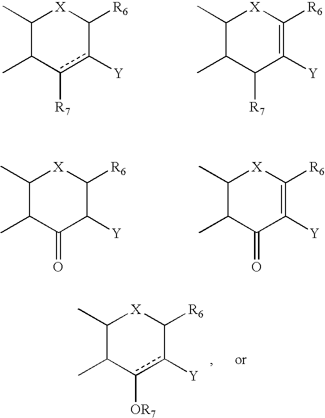
W, A and B taken together with the groups to which they are associated comprise 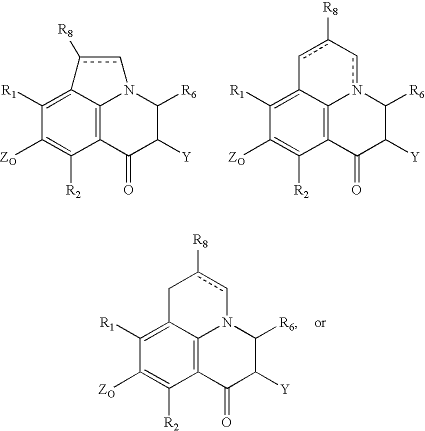
W and A taken together with the groups to which they are associated comprise 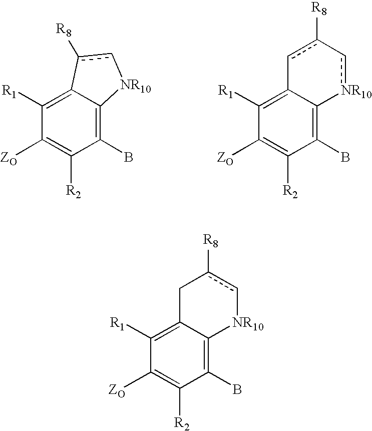
and B is 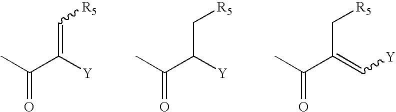
   wherein
R₃ is hydrogen, alkyl, aryl, arylalkyl, an amino acid, C(O)R₁₁ where R₁₁ is hydrogen alkyl, aryl, arylalkyl or an amino acid, or CO₂R₁₂ where R₁₂ is hydrogen, alkyl, haloalkyl, aryl or arylalkyl,
R₄ is hydrogen, alkyl or aryl,
or R₃ and R₄ taken together with the nitrogen to which they are attached comprise pyrrolidinyl or piperidinyl,
R₅ is hydrogen, C(O)R₁₁ where R₁₁ is as previously defined, or CO₂R₁₂ where R₁₂ is as previously defined,
R₆ is hydrogen, hydroxy, alkyl, aryl, amino, thio, NR₃R₄, COR₁₁ where R₁₁ is as previously defined, CO₂R₁₂ where R₁₂ is as previously defined or CONR₃R₄,
R₇ is hydrogen, C(O)R₁₁ where R₁₁ is as previously defined, alkyl, haloalkyl, aryl, arylalkyl or Si(R₁₃)₃ where each R₁₃ is independently hydrogen, alkyl or aryl,
R₈ is hydrogen, hydroxy, alkoxy or alkyl,
R₉ is alkyl, haloalkyl, aryl, arylalkyl, C(O)R₁₁ where R₁₁ is as previously defined, or Si(R₁₃)₃ where R₁₃ is as previously defined,
R₁₀ is hydrogen, alkyl, haloalkyl, amino, aryl, arylalkyl, an amino acid, alkylamino or dialkylamino,
   the drawing “ [symbol] ” represents either a single bond or a double bond,
T is independently hydrogen, alkyl or aryl,
X is O, NR₄ or S, and
Y is 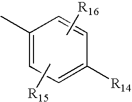
   wherein
R₁₄, R₁₅ and R₁₆ are independently hydrogen, hydroxy, OR₉, OC(O)R₁₀, OS(O)R₁₀, CHO, C(O)R₁₀, COOH, CO₂R₁₀, CONR₃R₄, alkyl, haloalkyl, aryl, arylalkyl, thio, alkylthio, amino, alkylamino, dialkylamino, nitro or halo, or any two of R₁₄, R₁₅ and R₁₆ are fused together to form a cyclic alkyl, aromatic or heteroaromatic structure,
   with the proviso that
when B is 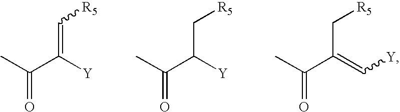
Y is phenyl, 4-hydroxyphenyl, 4-methoxyphenyl, 3-hydroxyphenyl, 3-methoxyphenyl, 3-hydroxy-4-methoxyphenyl, 4-hydroxy-3-methoxyphenyl, 3,4-dihydroxyphenyl or 3,4-dimethoxyphenyl, and
W and R₂ are hydrogen,
   then
R₁ is not hydrogen, hydroxy or methoxy, or R₁ and Z_{O} together with the carbon atoms to which they are attached are not cyclic boronates, carbonates, acetyls or ketals, and
when A and B taken together with the carbon atoms to which they are attached form a six-membered ring selected from 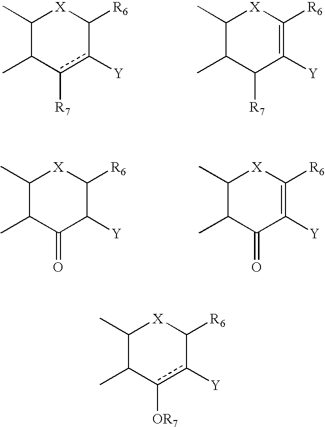
X is O,
Y is phenyl, 4-hydroxyphenyl, 4-methoxyphenyl, 3-hydroxyphenyl, 3-methoxyphenyl, 3-hydroxy-4-methoxyphenyl, 4-hydroxy-3-methoxyphenyl, 3,4-dihydroxyphenyl or 3,4-dimethoxyphenyl, and
W and R₂ are hydrogen,
   then
R₁ is not hydrogen, hydroxy or methoxy, or R₁ and Z_{O} together with the carbon atoms to which they are attached are not a cyclic boronate, carbonate, acetyl or ketal,
   or a pharmaceutically acceptable salt or prodrug thereof.

It has surprisingly been found by the inventors that the isoflavonoid derivatives of the general formula (I) have particular utility and effectiveness in the treatment, prophylaxis, amelioration defence against, and/or prevention of one of more of the following diseases and disorders (for convenience hereinafter referred to as the “therapeutic indications”):
(a) all forms of cancer (pre-malignant, benign and malignant) in all tissues of the body including breast cancer; uterine cancer; ovarian cancer; testicular cancer; large bowel cancer; endometrial cancer; prostatic cancer; uterine cancer. In this regard, the compounds may be used as the sole form of anti-cancer therapy or in combination with other forms of anti-cancer therapy including but not limited to radiotherapy and chemotherapy;
(b) diseases and disorders associated with inflammatory reactions of an abnormal or prolonged nature in any of the body's tissues including but not limited to rheumatoid arthritis, tendonitis, inflammatory bowel disease, ulcerative colitis, Crohn's Disease, sclerosing cholangitis;
(c) papulonodular skin lesions including but not limited to sarcoidosis, angiosarcoma, Kaposi's sarcome, Fabry's Disease
(d) papulosquamous skin lesions including but not limited to psoriasis, Bowen's Disease, and Reiter's Disease;
(e) actinic damage characterized by degenerative changes in the skin including but not limited to solar keratosis, photosensitivity diseases, and wrinkling;
(f) diseases and disorders associated with abnormal angiogenesis affecting any tissue within the body including but not limited to hemangiomas and telangiectasia;
(g) proliferative disorders of bone marrow including but not limited to megaloblastic disease, myelodysplastic syndromes, polycythemia vera, thrombocytosis and myelofibrosis;
(h) autoimmune disease characterized by abnormal immunological responses including but not limited to multiple sclerosis, Type 1 diabetes, systemic lupus erythematosis, and biliary cirrhosis;
(i) neurodegenerative diseases and disorders characterised by degenerative changes in the structure of the neurological system including but not limited to Parkinson's Disease, Alzheimer's Disease, muscular dystrophy, Lou-Gehrig Disease, motorneurone disease;
(j) diseases and disorders associated with degenerative changes within the walls of blood vessels including but not limited to atherosclerosis, stenosis, restenosis, atheroma, coronary artery disease, stroke, myocardial infarction, hypertensive vascular disease, malignant hypertension, thromboangiitis obliterans, fibromuscular dysplasia;
(k) diseases and disorders associated with abnormal immunological responses including but limited to dermatomyositis and scleroderma;
(l) diseases and disorders associated with degenerative changes within the eye including but not limited to cataracts, macular degeneration, retinal atrophy.

In particular the isoflavonoid derivatives also surprisingly have been found to have a potent effect on the production and function of reproductive hormones such as estrogens and androgens. As a result of this, these compounds may be used in the treatment and prevention of one or more of the following disorders and diseases:
(a) conditions in women associated with abnormal estrogen/androgen balance including but not limited to cyclical mastalgia, acne, dysmenorrhoea, uterine fibroids, endometriosis, ovarian cysts, premenstrual syndrome, acute menopause symptoms, osteoporosis, senile dementia, infertility; and
(b) conditions in men associated with abnormal estrogen/androgen balance including but not limited to benign prostatic hypertrophy, infertility, gynecomastia, alopecia hereditaria and various other forms of baldness.

Thus, according to a second aspect of the present invention there is provided a method for the treatment, prophylaxis or amelioration of a disease or disorder which method includes the step of administering a therapeutically effective amount of one or more compounds of formula (I) to a subject.

According to a third aspect of the present invention there is provided the use of one or more compounds of formula (I) in the manufacture of a medicament for the treatment of disease or disorder.

According to a forth aspect of the present invention there is provided the use of one or more compounds selected from formula (I) for the treatment, amelioration, defence against, prophylaxis and/or prevention of abnormal estrogen/androgen balance or a condition resulting from said abnormal balance in men or women.

According to a fifth aspect of the present invention there is provided the use of one or more compounds selected from formula (I) in the manufacture of a medicament for the treatment, amelioration, defence against, prophylaxis and/or prevention of abnormal estrogen/androgen balance or a condition resulting from said abnormal balance in men or women.

According to a sixth aspect of the present invention there is provided an agent for the treatment, prophylaxis or amelioration of a disease or disorder which agent comprises one or more compounds of formula (I).

According to a seventh aspect of the present invention there is provided a pharmaceutical composition which comprises one or more compounds of formula (I) in association with one or more pharmaceutical carriers, excipients, auxiliaries and/or diluents.

According to an eighth aspect of the present invention there is provided a drink or food-stuff, which contains one or more compounds of formula (I).

According to a ninth aspect of the present invention there is provided a microbial culture or a food-stuff containing one or more microbial strains which microorganisms produce one or more compounds of formula (I).

According to an tenth aspect of the present invention there is provided one or more microorganisms which produce one or more compounds of formula (I). Preferably the microorganism is a purified culture, which may be admixed and/or administered with one or more other cultures which product compounds of formula (I).

These and other aspects of the invention will become evident from the description and claims which follow.

Throughout this specification and the claims which follow, unless the text requires otherwise, the word “comprise”, and variations such as “comprises” or “comprising”, will be understood to imply the inclusion of a stated integer or step or group of integers or steps but not the exclusion of any other integer or step or group of integers or steps.

### DETAILED DESCRIPTION OF THE INVENTION

The term “isoflavone” as used herein is to be taken broadly to include ring-fused benzopyran molecules having a pendent phenyl group from the pyran ring based on a 1,2-diphenylpropane system and to ring-opened benzopyran molecules where the pyran oxygen may also be a heteratom selected from nitrogen and sulfur. Thus, the classes of compounds generally referred to as isoflavones, isoflavenes, isoflavans, isoflavanones, isoflavanols and the like are generically referred to herein as isoflavones, isoflavone derivatives or isoflavonoid molecules, compounds or derivatives.

The term “alkyl” is taken to include straight chain, branched chain and cyclic (in the case of 5 carbons or greater) saturated alkyl groups of 1 to 10 carbon atoms, preferably from 1 to 6 carbon atoms, such as methyl, ethyl, propyl, isopropyl, butyl, isobutyl, secbutyl, tertiary butyl, pentyl, cyclopentyl, and the like. The alkyl group is more preferably methyl, ethyl, propyl or isopropyl. The alkyl group may optionally be substituted by one or more of fluorine, chlorine, bromine, iodine, carboxyl, C₁-C₄-alkoxycarbonyl, C₁-C₄-alkylamino-carbonyl, di-(C₁-C₄-alkyl)-amino-carbonyl, hydroxyl, C₁-C₄-alkoxy, formyloxy, C₁-C₄-alkyl-carbonyloxy, C₁-C₄-alkylthio, C₃-C₆-cycloalkyl or phenyl.

The term “alkenyl” is taken to include straight chain, branched chain and cyclic (in the case of 5 carbons or greater) hydrocarbons of 2 to 10 carbon atoms, preferably 2 to 6 carbon atoms, with at lease one double bond such as ethenyl, 1-propenyl, 2-propenyl, 1-butenyl, 2-butenyl, 2-methyl-1-peopenyl, 2-methyl-2-propenyl, and the like. The alkenyl group is more preferably ethenyl, 1-propenyl or 2-propenyl. The alkenyl groups may optionally be substituted by one or more of fluorine, chlorine, bromine, iodine, carboxyl, C₁-C₄-alkoxycarbonyl, C₁-C₄-alkylamino-carbonyl, di-(C₁-C₄-alkyl)-amino-carbonyl, hydroxyl, C₁-C₄-alkoxy, formyloxy, C₁-C₄-alkyl-carbonyloxy, C₁-C₄-alkylthio, C₃-C₆-cycloalkyl or phenyl.

The term “alkynyl” is taken to include both straight chain and branched chain hydrocarbons of 2 to 10 carbon atoms, preferably 2 to 6 carbon atoms, with at least one triple bond such as ethynyl, 1-propynyl, 2-propynyl, 1-butynyl, 2-butynyl, and the like. The alkynyl group is more preferably ethynyl, 1-propynyl or 2-propynyl. The alkynyl group may optionally be substituted by one or more of fluorine, chlorine, bromine, iodine, carboxyl, C₁-C₄-alkoxycarbonyl, C₁-C₄-alkylamino-carbonyl, di-(C₁-C₄-alkyl)-amino-carbonyl, hydroxyl, C₁-C₄-alkoxy, formyloxy, C₁-C₄-alkyl-carbonyloxy, C₁-C₄-alkylthio, C₃-C₆-cycloalkyl or phenyl.

The term “aryl” is taken to include phenyl, biphenyl and naphthyl and may be optionally substituted by one or more C₁-C₄-alkyl, hydroxy, C₁-C₄-alkoxy, carbonyl, C₁-C₄-alkoxycarbonyl, C₁-C₄-alkylcarbonyloxy or halo.

The term “heteroaryl” is taken to include five-membered and six-membered rings which include at least one oxygen, sulfur or nitrogen in the ring, which rings may be optionally fused to other aryl or heteroaryl rings including but not limited to furyl, pyridyl, pyrimidyl, thienyl, imidazolyl, tetrazolyl, pyrazinyl, benzofuranyl, benzothiophenyl, quinolyl, isopuinolyl, purinyl, morpholinyl, oxazolyl, thiazolyl, pyrrolyl, xanthinyl, purine, thymine, cytosine, uracil, and isoxazolyl. The heteroaromatic group can be optionally substituted by one or more of fluorine, chlorine, bromine, iodine, carboxyl, C₁-C₄-alkoxycarbonyl, C₁-C₄-alkylamino-carbonyl, di-(C₁-C₄-alkyl)-amino-carbonyl, hydroxyl, C₁-C₄-alkoxy, formyloxy, C₁-C₄-alkyl-carbonyloxy, C₁-C₄-alkylthio, C₃-C₆-cycloalkyl or phenyl. The heteroaromatic can be partially or totally hydrogenated as desired.

The term “halo” is taken to include fluoro, chloro, bromo and iodo, preferably fluoro and chloro, more preferably fluoro. Reference to for example “haloalkyl” will include monohalogenated, dihalogenated and up to perhalogenated alkyl groups. Preferred haloalkyl groups are trifluoromethyl and pentafluoroethyl.

The term “pharmaceutically acceptable salt” refers to an organic or inorganic moiety that carries a charge and that can be administered in association with a pharmaceutical agent, for example, as a counter-cation or counter-anion in a salt. Pharmaceutically acceptable cations are known to those of skilled in the art, and include but are not limited to sodium, potassium, calcium, zinc and quaternary amine. Pharmaceutically acceptable anions are known to those of skill in the art, and include but are not limited to chloride, acetate, citrate, bicarbonate and carbonate.

The term “pharmaceutically acceptable derivative” or “prodrug” refers to a derivative of the active compound that upon administration to the recipient is capable of providing directly or indirectly, the parent compound or metabolite, or that exhibits activity itself.

As used herein, the terms “treatment”, “prophylaxis” or “prevention”, “amelioration” and the like are to be considered in their broadest context. In particular, the term “treatment” does not necessarily imply that an animal is treated until total recovery. Accordingly, “treatment” includes amelioration of the symptoms or severity of a particular condition or preventing or otherwise reducing the risk of developing a particular condition.

The invention in particular relates to compounds of the general formulae (II)-(VIII): 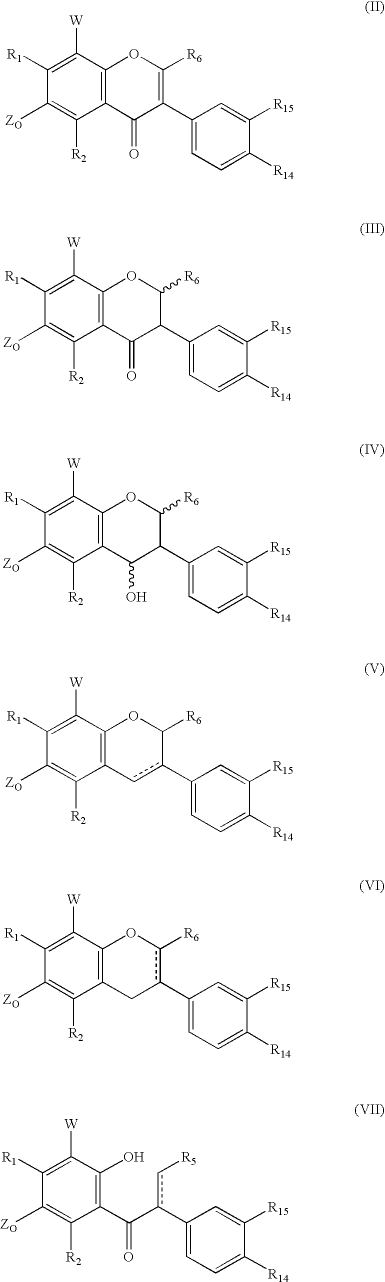 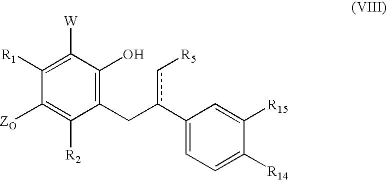
in which
R₁, R₂, R₅, R₆, R₁₄, R¹⁵, W and Z_{O} are as defined above
   more preferably
R₁, R₂, R₁₄, R₁₅, and W are independently hydrogen, hydroxy, OR₉, OC(O)R₁₀, C(O)R₁₀, COOH, CO₂R₁₀, alkyl, haloalkyl, arylalkyl, aryl, thio, alkylthio, amino, alkylamino, dialkylamino, nitro or halo,
Z_{O} is hydroxy,
R₅ is hydrogen, C(O)R₁₁ where R₁₁ is hydrogen, alkyl, aryl, or an amino acid, or CO₂R₁₂ where R₁₂ is hydrogen, alkyl or aryl,
R₆ is hydrogen, hydroxy, alkyl, aryl, COR₁₁ where R₁₁ is as previously defined, or CO₂R₁₂ where R₁₂ is as previously defined,
R₉ is alkyl, haloalkyl, arylalkyl, or C(O)R₁₁ where R₁₁ is as previously defined, and
R₁₀ is hydrogen, alkyl, amino, aryl, an amino acid, alkylamino or dialkylamino,
   more preferably
R₁ and R₁₄ are independently hydroxy, OR₉, OC(O)R₁₀ or halo,
R₂, R₁₅, and W are independently hydrogen, hydroxy, OR₉, OC(O)R₁₀, C(O)R₁₀, COOH, CO₂R₁₀, alkyl, haloalkyl, or halo,
Z_{O} is hydroxy,
R₅ is hydrogen, C(O)R₁₁ where R₁₁ is hydrogen or alkyl, or CO₂R₁₂ where R₁₂ is hydrogen or alkyl,
R₆ is hydrogen or hydroxy,
R₉ is alkyl, arylalkyl or C(O)R₁₁ where R₁₁ is as previously defined, and
R₁₀ is hydrogen or alkyl,
   and more preferably
R₁ and R₁₄ are independently hydroxy, methoxy, benzyloxy, acetyloxy or chloro,
R₂, R₁₅, and W are independently hydrogen, hydroxy, methoxy, benzyloxy, acetyloxy, methyl, trifluoromethyl or chloro,
Z_{O} is hydroxy,
R₅ is hydrogen or CO₂R₁₂ where R₁₂ is hydrogen or methyl, and
R₆ is hydrogen.

Particularly preferred compounds of the present invention are selected from:

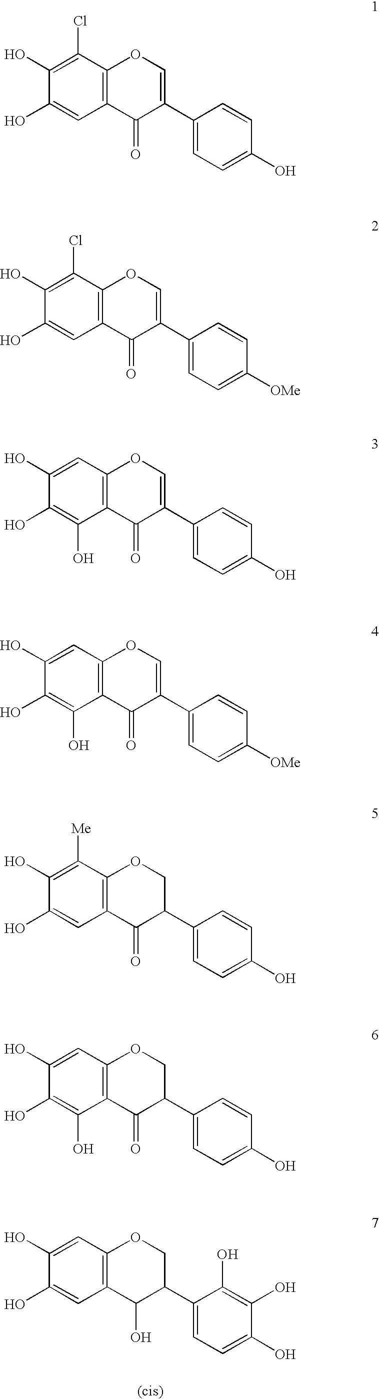

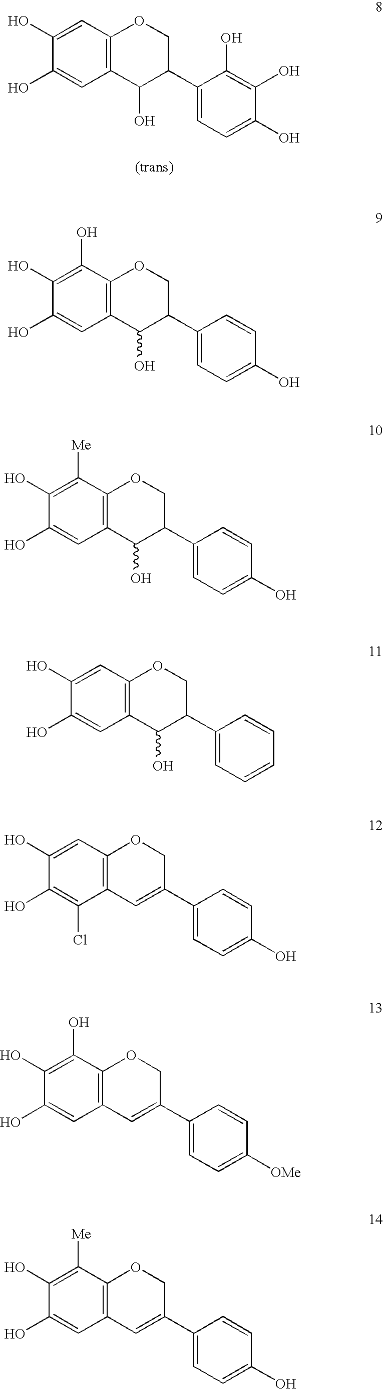

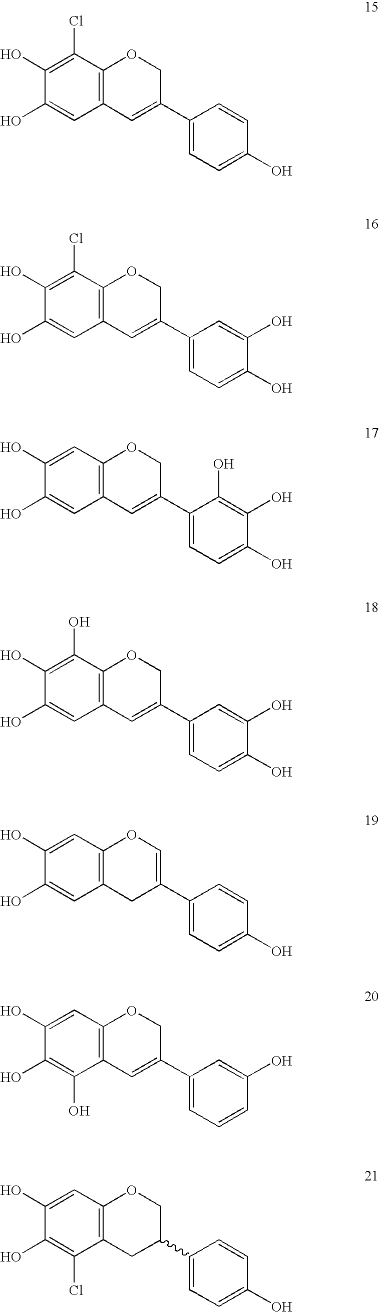

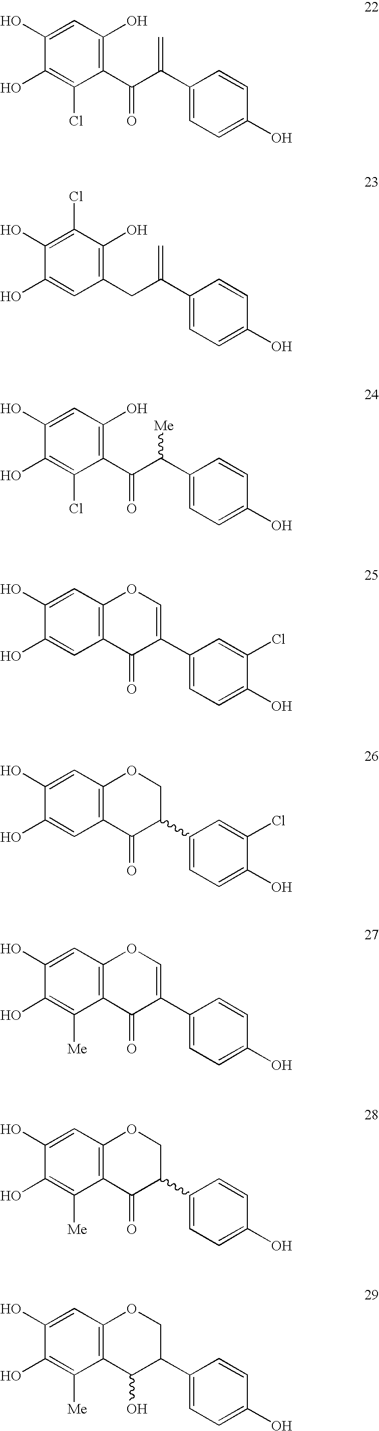

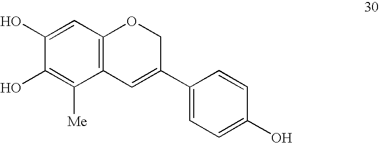

The preferred compounds of the present invention also include all derivatives with physiologically cleavable leaving groups that can be cleaved in vivo from the isoflavone or derivative molecule to which it is attached. The leaving groups include acyl, phosphate, sulfate, sulfonate, and preferably are mono-, di- and per-acyl oxy-substituted compounds, where one or more of the pendant hydroxy groups are protected by an acyl group, preferably an acetyl group. Typically acyloxy substituted isoflavones and derivatives thereof are readily cleavable to the corresponding hydroxy substituted compounds. In addition, the protection of functional groups on the isoflavone compounds and derivatives of the present invention can be carried out by well established methods in the art, for example as described in Protective Groups in Organic Syntheses, T. W. Greene, John Wiley & Sons, New York, 1981.

Chemical and functional equivalents of a particular isoflavone should be understood as molecules exhibiting any one of more of the functional activities of the isoflavone and may be derived from any source such as being chemically synthesised or identified via screening processes such as natural product screening.

Compounds of the present invention have particular application in the treatment of diseases associated with or resulting from estrogenic effects, androgenic effects, vasodilatory and spasmodic effects, inflammatory effects and oxidative effects.

The amount of one or more compounds of formula I which is required in a therapeutic treatment according to the invention will depend upon a number of factors, which include the specific application, the nature of the particular compound used, the condition being treated, the mode of administration and the condition of the patient. Compounds of formula I may be administered in a manner and amount as is conventionally practised. See, for example, Goodman and Gilman, The Pharmacological Basis of Therapeutics, 1299 (7th Edition, 1985). The specific dosage utilised will depend upon the condition being treated, the state of the subject, the route of administration and other well known factors as indicated above. In general, a daily dose per patient may be in the range of 0.1 mg to 2 g; typically from 0.5 mg to 1 g; preferably from 50 mg to 200 mg.

The production of pharmaceutical compositions for the treatment of the therapeutic indications herein described are typically prepared by admixture of the compounds of the invention (for convenience hereafter referred to as the “active compounds”) with one or more pharmaceutically or veterinarially acceptable carriers and/or excipients as are well known in the art.

The carrier must, of course, be acceptable in the sense of being compatible with any other ingredients in the formulation and must not be deleterious to the subject. The carrier or excipient may be a solid or a liquid, or both, and is preferably formulated with the compound as a unit-dose, for example, a tablet, which may contain from 0.5% to 59% by weight of the active compound, or up to 100% by weight of the active compound. One or more active compounds may be incorporated in the formulations of the invention, which may be prepared by any of the well known techniques of pharmacy consisting essentially of admixing the components, optionally including one or more accessory ingredients.

The formulations of the invention include those suitable for oral, rectal, optical, buccal (for example, sublingual), parenteral (for example, subcutaneous, intramuscular, intradermal, or intravenous) and transdermal administration, although the most suitable route in any given case will depend on the nature and severity of the condition being treated and on the nature of the particular active compound which is being used.

Formulation suitable for oral administration may be presented in discrete units, such as capsules, sachets, lozenges, or tablets, each containing a predetermined amount of the active compound; as a powder or granules; as a solution or a suspension in an aqueous or non-aqueous liquid; or as an oil-in-water or water-in-oil emulsion. Such formulations may be prepared by any suitable method of pharmacy which includes the step of bringing into association the active compound and a suitable carrier (which may contain one or more accessory ingredients as noted above). In general, the formulations of the invention are prepared by uniformly and intimately admixing the active compound with a liquid or finely divided solid carrier, or both, and then, if necessary, shaping the resulting mixture such as to form a unit dosage. For example, a tablet may be prepared by compressing or moulding a powder or granules containing the active compound, optionally with one or more accessory ingredients. Compressed tablets may be prepared by compressing, in a suitable machine, the compound of the free-flowing, such as a powder or granules optionally mixed with a binder, lubricant, inert diluent, and/or surface active/dispersing agent(s). Moulded tablets may be made by moulding, in a suitable machine, the powdered compound moistened with an inert liquid binder.

Formulations suitable for buccal (sublingual) administration include lozenges comprising the active compound in a flavoured base, usually sucrose and acacia or tragacanth; and pastilles comprising the compound in an inert base such as gelatin and glycerin or sucrose and acacia.

Compositions of the present invention suitable for parenteral administration conveniently comprise sterile aqueous preparations of the active compounds, which preparations are preferably isotonic with the blood of the intended recipient. These preparations are preferably administered intravenously, although administration may also be effected by means of subcutaneous, intramuscular, or intradermal injection. Such preparations may conveniently be prepared by admixing the compound with water or a glycine buffer and rendering the resulting solution sterile and isotonic with the blood. Injectable formulations according to the invention generally contain from 0.1% to 60% w/v of active compound and are administered at a rate of 0.1 ml/minute/kg.

Formulations suitable for rectal administration are preferably presented as unit dose suppositories. These may be prepared by admixing the active compound with one or more conventional solid carriers, for example, cocoa butter, and then shaping the resulting mixture.

Formulations or compositions suitable for topical administration to the skin preferably take the form of an ointment, cream, lotion, paste, gel, spray, aerosol, or oil. Carriers which may be used include Vaseline, lanoline, polyethylene glycols, alcohols, and combination of two or more thereof. The active compound is generally present at a concentration of from 0.1% to 0.5% w/w, for example, from 0.5% to 2% w/w. Examples of such compositions include cosmetic skin creams.

Formulations suitable for transdermal administration may be presented as discrete patches adapted to remain in intimate contact with the epidermis of the recipient for a prolonged period of time. Such patches suitably contain the active compound as an optionally buffered aqueous solution of, for example, 0.1 M to 0.2 M concentration with respect to the said active compound.

Formulations suitable for transdermal administration may also be delivered by iontophoresis (see, for example, Pharmaceutical Research 3 (6), 318 (1986)) and typically take the form of an optionally buffered aqueous solution of the active compound. Suitable formulations comprise citrate or bis/tris buffer (pH 6) or ethanol/water and contain from 0.1 M to 0.2 M active ingredient.

The active compounds may be provided in the form of food stuffs, such as being added to, admixed into, coated, combined or otherwise added to a food stuff. The term food stuff is used in its widest possible sense and includes liquid formulations such as drinks including dairy products and other foods, such as health bars, desserts, etc. Food formulations containing compounds of the invention can be readily prepared according to standard practices.

Compounds of the present invention have potent antioxidant activity and thus find wide application in pharmaceutical and veterinary uses, in cosmetics such as skin creams to prevent skin ageing, in sun screens, in foods, health drinks, shampoos, and the like.

It has surprisingly been found that compounds of the formula I interact synergisticly with vitamin E to protect lipids, proteins and other biological molecules from oxidation.

Accordingly a further aspect of this invention provides a composition comprising one or more compounds of formula I, vitamin E, and optionally a pharmaceutically, veterinarily or cosmetically acceptable carriers and/or excipients.

Therapeutic methods, uses and compositions may be for administration to humans or animals, such as companion and domestic animals (such as dogs and cats), birds (such as chickens, turkeys, ducks), livestock animals (such as cattle, sheep, pigs and goats) and the like.

Compounds of formula I may be prepared by standard methods known to those skilled in the art. Suitable methods may be found in, for example, International Patent Applications WO 98/08503 and WO 00/49009 which are incorporated herein in their entirety by reference. Methods which may be employed by those skilled in the art of chemical synthesis for constructing the general ring structures depicted in formulae I and II are depicted in schemes 1-8 below. Chemical functional group protection, deprotection, synthons and other techniques known to those skilled in the art may be used where appropriate in the synthesis of the compounds of the present invention. In the formulae depicted in the schemes below the moities R₁, R₂, R₆, R₈, R₁₄, R₁₅, R₁₆, W and X are as defined above. The hydroxy moiety Z_{O} may also be protected, deprotected or derived from a synthon as appropriate during the synthesis or administration of the compounds of the present invention. Reduction of the isoflavone derivatives may be effected by procedures well known to those skilled in the art including sodium borohydride reduction, and hydration over metal catalysts such as Pd/C, Pd/CaCO₃ and Platinum(IV)oxide (Adam's catalyst) in protic or aprotic solvents. The end products and isomeric ratios can be varied depending on the catalyst/solvent system chosen. The schemes depicted below are not to be considered limiting on the scope of the invention described herein.

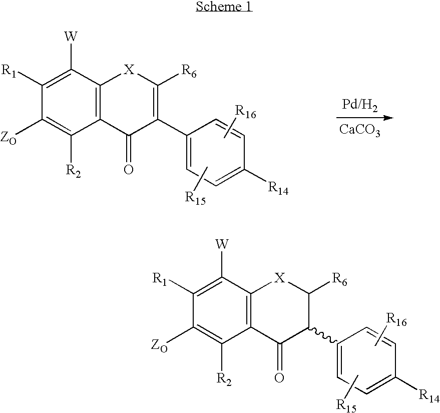

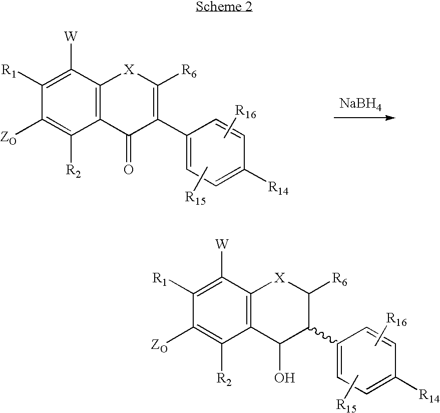

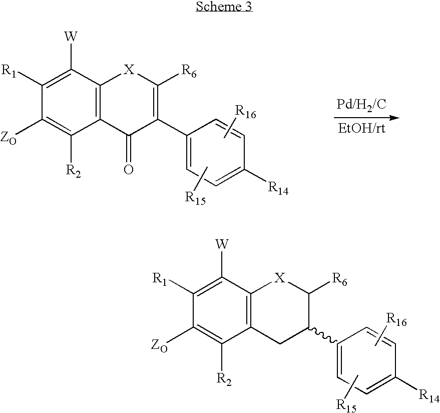

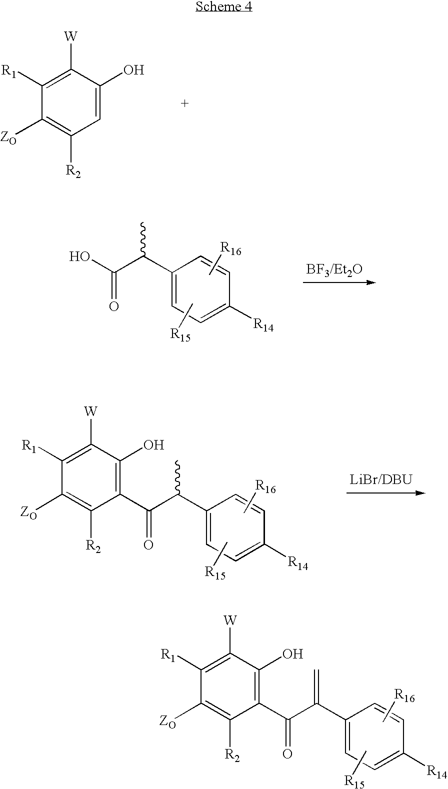

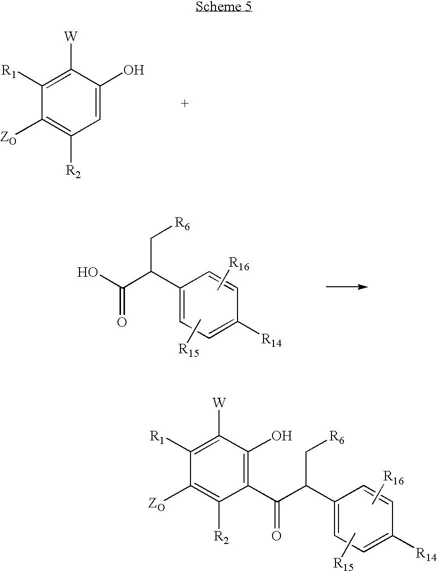

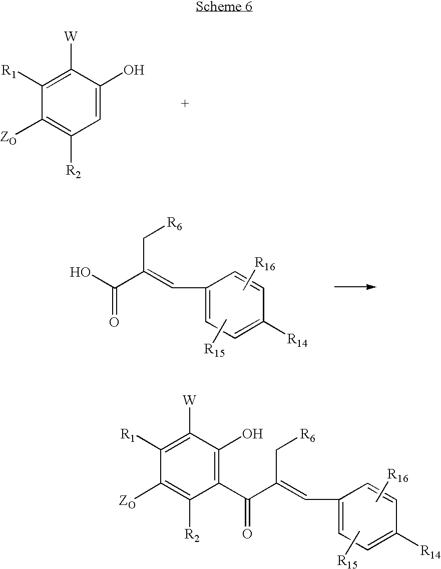

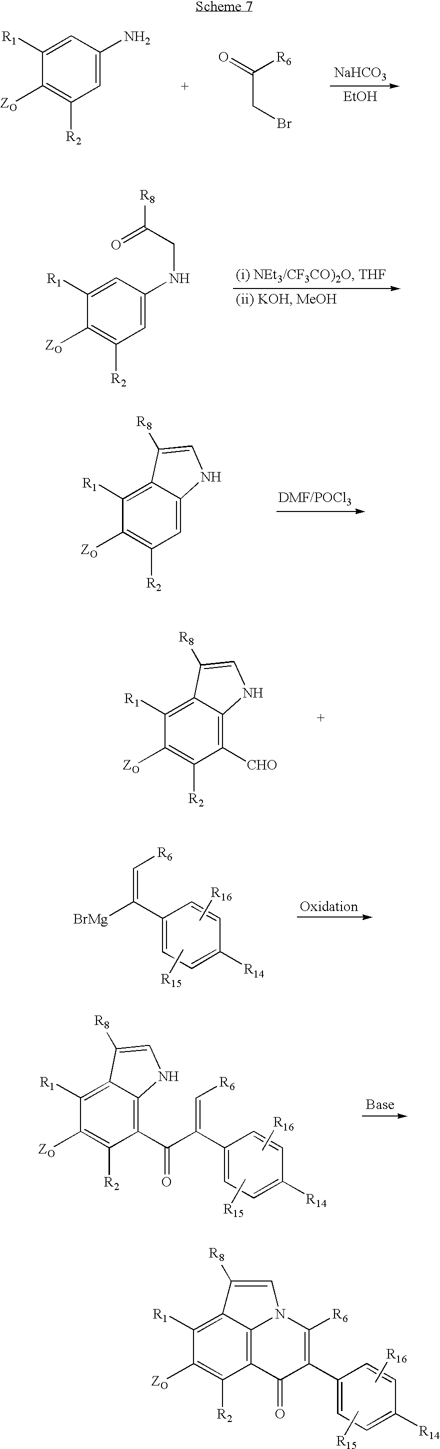

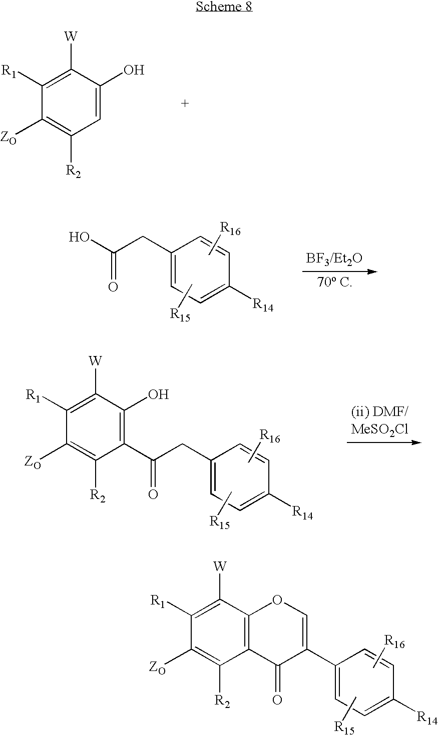

The invention will now be further described by the following non-limiting examples.

### EXAMPLE 1

General Syntheses of Substituted Isoflavones

8-Chloro-4′,6,7-trihydroxyisoflavone (1) was synthesised by the general method of condensing 3-chloro-1,2,4-benzenetriol with 4-hydroxyphenylacetic acid to afford 2-chloro-2,4,5,4′-tetrahydroxydeoxybenzoin according to Scheme 8. Cyclisation of the intermediate deoxybenzoin was achieved by treatment with dimethylformamide and methanesulfonyl chloride in the presence of boron triflouride etherate to afford compound (1).

In a similar manner numerous other substituted isoflavones and derivatives thereof of general formula (I) and formulae (II)-(VIII) and compounds (2)-(30) can also be synthesised by varying the substitution pattern and/or protecting groups on the phenol derivatives or phenylacetic acid groups. For example starting with 6-methyl-1,2,4-benzenetriol affords 4′,6,7-trihydroxy-5-methylisoflavone (27); whilst use of 3-hydroxy phenyl acetic acid in the general synthetic method affords 3′-hydroxy isoflavone derivatives, such as compound (20).

It will be appreciated by those skilled in the art that protecting groups may be utilised in the synthetic methods described as appropriate. For example, vicinal hydroxy groups can be protected as cyclic ketals, acetyls, boronates and carbonates according to standard methods known in the art (see for example March, Advanced Organic Chemistry, 3rd Ed., 1985, John Wiley & Sons). Additionally or alternatively well known protection and deprotection methods of functional group chemistry or synthons may be employed (see Green, ibid. or March, ibid., or references cited therein).

As an example, the starting phenol from Scheme 8 may be first protected as an n-butyl boronate: 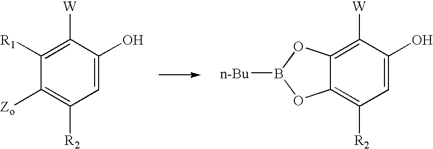
and then deprotected as required during the synthesis of the compounds of formula (I).

### EXAMPLE 2

The binding affinity of various compounds of the invention for both subtypes of the estrogen receptor is determined using the “Estrogen Receptor Alpha or Beta Competitor Assay Core HTS Kit” supplied by Panvera Corporation (Product No. P2614/2615). Many of the exemplified and named compounds show good competitive binding to the estrogen receptors ER alpha and ER beta.

The results show that the compounds of the present invention have particular application in the treatment, prophylaxis or amelioration of diseases associated with or resulting from estrogenic effects, androgenic effects, vasodilatory and spasmodic effects, inflammatory effects and oxidative effects.

The invention has been described herein, with reference to certain preferred embodiments, in order to enable the reader to practice the invention without undue experimentation. However, a person having ordinary skill in the art will readily recognise that many of the components and parameters may be varied or modified to a certain extent without departing from the scope of the invention. Furthermore, titles, headings, or the like are provided to enhance the reader's comprehension of this document, and should not be read as limiting the scope of the present invention.

The entire disclosures of all applications, patents and publications, cited herein, if any, are hereby incorporated by reference.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification individually or collectively, and any and all combinations of any two or more of said steps or features.

The reference to any prior art in this specification is not, and should not be taken as, an acknowledgment or any form of suggestion that that prior art forms part of the common general knowledge in the field of endeavour.

1. An isoflavone compound or analogue thereof of the general formula (I): 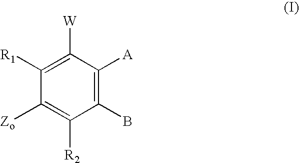
in which
R₁ and R₂ are independently hydrogen, hydroxy, OR₉, OC(O)R₁₀, OS(O)R₁₀, CHO, C(O)R₁₀, COOH, CO₂R₁₀, CONR₃R₄, alkyl, haloalkyl, aryl, arylalkyl, thio, alkylthio, amino, alkylamino, dialkylamino, nitro or halo, and
Z_{O} is hydroxy, or
R₂ is as previously defined, and R₁ and Z_{O} taken together with the carbon atoms to which they are attached form a five-membered ring selected from 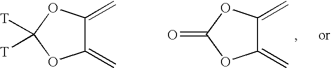
R₁ is as previously defined, and R₂ and Z_{O} taken together with the carbon atoms to which they are attached form a five-membered ring selected from 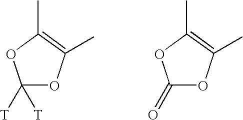
and
W is R₁, A is hydrogen, hydroxy, NR₃R₄ or thio, and B is selected from 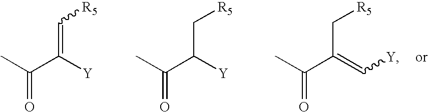
W is R₁, and A and B taken together with the carbon atoms to which they are attached form a six-membered ring selected from 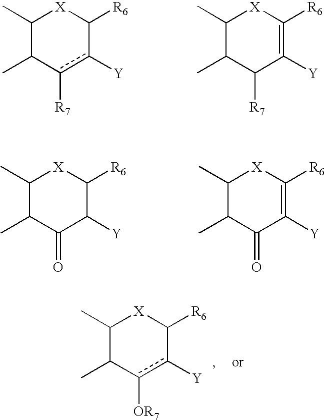
W, A and B taken together with the groups to which they are associated comprise 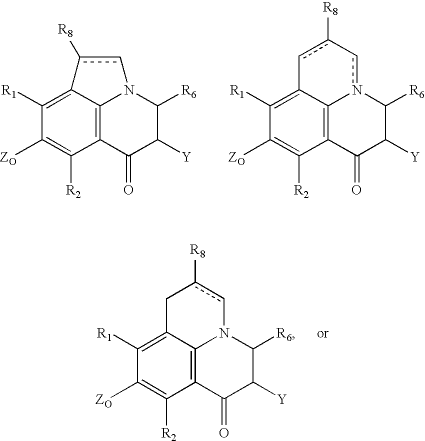
W and A taken together with the groups to which they are associated comprise 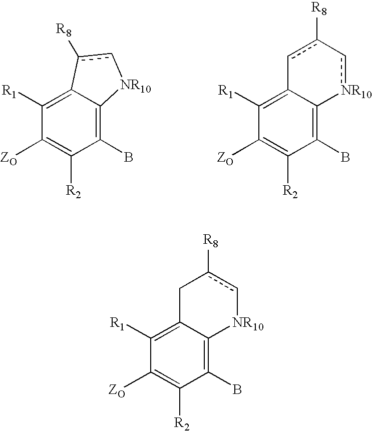
and B is 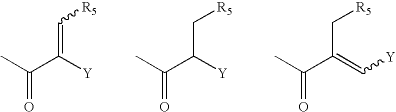
wherein
R₃ is hydrogen, alkyl, aryl, arylalkyl, an amino acid, C(O)R₁₁ where R₁₁ is hydrogen alkyl, aryl, arylalkyl or an amino acid, or CO₂R₁₂ where R₁₂ is hydrogen, alkyl, haloalkyl, aryl or arylalkyl,
R₄ is hydrogen, alkyl or aryl,
or R₃ and R₄ taken together with the nitrogen to which they are attached comprise pyrrolidinyl or piperidinyl,
R₅ is hydrogen, C(O)R₁₁ where R₁₁ is as previously defined, or CO₂R₁₂ where R₁₂ is as previously defined,
R₆ is hydrogen, hydroxy, alkyl, aryl, amino, thio, NR₃R₄, COR₁₁ where R₁₁ is as previously defined, CO₂R₁₂ where R₁₂ is as previously defined or CONR₃R₄,
R₇ is hydrogen, C(O)R₁₁ where R₁₁ is as previously defined, alkyl, haloalkyl, aryl, arylalkyl or Si(R₁₃)₃ where each R₁₃ is independently hydrogen, alkyl or aryl,
R₈ is hydrogen, hydroxy, alkoxy or alkyl,
R₉ is alkyl, haloalkyl, aryl, arylalkyl, C(O)R₁₁ where R₁₁ is as previously defined, or Si(R₁₃)₃ where R₁₃ is as previously defined,
R₁₀ is hydrogen, alkyl, haloalkyl, amino, aryl, arylalkyl, an amino acid, alkylamino or dialkylamino,
the drawing “ [symbol] ” represents either a single bond or a double bond,
T is independently hydrogen, alkyl or aryl,
X is O, NR₄ or S, and
Y is 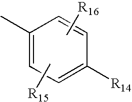
wherein
R₁₄, R₁₅ and R₁₆ are independently hydrogen, hydroxy, OR₉, OC(O)R₁₀, OS(O)R₁₀, CHO, C(O)R₁₀, COOH, CO₂R₁₀, CONR₃R₄, alkyl, haloalkyl, aryl, arylalkyl, thio, alkylthio, amino, alkylamino, dialkylamino, nitro or halo, or any two of R₁₄, R₁₅ and R₁₆ are fused together to form a cyclic alkyl, aromatic or heteroaromatic structure,
with the proviso that
when B is 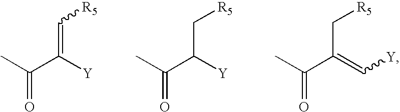
Y is phenyl, 4-hydroxyphenyl, 4-methoxyphenyl, 3-hydroxyphenyl, 3-methoxyphenyl, 3-hydroxy-4-methoxyphenyl, 4-hydroxy-3-methoxyphenyl, 3,4-dihydroxyphenyl or 3,4-dimethoxyphenyl, and
W and R₂ are hydrogen,
then
R₁ is not hydrogen, hydroxy or methoxy, or R₁ and Z_{O} together with the carbon atoms to which they are attached are not cyclic boronates, carbonates, acetyls or ketals, and
when A and B taken together with the carbon atoms to which they are attached form a six-membered ring selected from 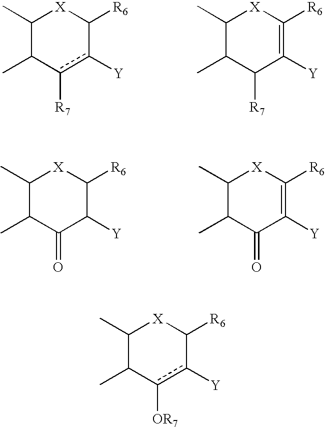
X is O,
Y is phenyl, 4-hydroxyphenyl, 4-methoxyphenyl, 3-hydroxyphenyl, 3-methoxyphenyl, 3-hydroxy-4-methoxyphenyl, 4-hydroxy-3-methoxyphenyl, 3,4-dihydroxyphenyl or 3,4-dimethoxyphenyl, and
W and R₂ are hydrogen,
then
R₁ is not hydrogen, hydroxy or methoxy, or R₁ and Z_{O} together with the carbon atoms to which they are attached are not a cyclic boronate, carbonate, acetyl or ketal,
or a pharmaceutically acceptable salt or prodrug thereof.

2. A compound of formula (I) according to claim 1 selected from a compound of the general formulae (II)-(VIII)
in which
R₁, R₂, R₅, R₆, R₁₄, R₁₅, W and Z_{O} are as defined in claim 1.

3. A compound of claim 2 wherein
R₁, R₂, R₁₄, R₁₅, and W are independently hydrogen, hydroxy, OR₉, OC(O)R₁₀, C(O)R₁₀, COOH, CO₂R₁₀, alkyl, haloalkyl, arylalkyl, aryl, thio, alkylthio, amino, alkylamino, dialkylamino, nitro or halo,
Z_{O} is hydroxy,
R₅ is hydrogen, C(O)R₁₁ where R₁₁ is hydrogen, alkyl, aryl, or an amino acid, or CO₂R₁₂ where R₁₂ is hydrogen, alkyl or aryl,
R₆ is hydrogen, hydroxy, alkyl, aryl, COR₁₁ where R₁₁ is as previously defined, or CO₂R₁₂ where R₁₂ is as previously defined,
R₉ is alkyl, haloalkyl, arylalkyl, or C(O)R₁₁ where R₁₁ is as previously defined, and
R₁₀ is hydrogen, alkyl, amino, aryl, an amino acid, alkylamino or dialkylamino.

4. A compound of claim 3 wherein
R₁ and R₁₄ are independently hydroxy, OR₉, OC(O)R₁₀ or halo,
R₂, R₁₅, and W are independently hydrogen, hydroxy, OR₉, OC(O)R₁₀, C(O)R₁₀, COOH, CO₂R₁₀, alkyl, haloalkyl, or halo,
Z_{O} is hydroxy,
R₅ is hydrogen, C(O)R₁₁ where R₁₁ is hydrogen or alkyl, or CO₂R₁₂ where R₁₂ is hydrogen or alkyl,
R₆ is hydrogen or hydroxy,
R₉ is alkyl, arylalkyl or C(O)R₁₁ where R₁₁ is as previously defined, and
R₁₀ is hydrogen or alkyl.

5. A compound of claim 4 wherein
R₁ and R₁₄ are independently hydroxy, methoxy, benzyloxy, acetyloxy or chloro,
R₂, R₁₅, and W are independently hydrogen, hydroxy, methoxy, benzyloxy, acetyloxy, methyl, trifluoromethyl or chloro,
Z_{O} is hydroxy,
R₅ is hydrogen or CO₂R₁₂ where R₁₂ is hydrogen or methyl, and
R₆ is hydrogen.

6. A compound of formula (I) selected from the compounds numbered 1 to 30 as herein described or a pharmaceutically acceptable salt or prodrug thereof.

7. A method for the treatment, prophylaxis, amelioration, defence against, and/or prevention of one or more diseases and disorders which comprises administering to a subject a therapeutically effective amount of one or more compounds selected from formula (I).

8. A method of claim 7 wherein the diseases and disorders are selected from:
(a) all forms of cancer (pre-malignant, benign and malignant) in all tissues of the body including breast cancer; uterine cancer; ovarian cancer; testicular cancer; large bowel cancer; endometrial cancer; prostatic cancer; and uterine cancer;
(b) diseases and disorders associated with inflammatory reactions of an abnormal or prolonged nature in any of the body's tissues including rheumatoid arthritis, tendonitis, inflammatory bowel disease, ulcerative colitis, Crohn's Disease, and sclerosing cholangitis;
(c) papulonodular skin lesions including sarcoidosis, angiosarcoma, Kaposi's sarcome, and Fabry's Disease
(d) papulosquamous skin lesions including psoriasis, Bowen's Disease, and Reiter's Disease;
(e) actinic damage characterized by degenerative changes in the skin including solar keratosis, photosensitivity diseases, and wrinkling;
(f) diseases and disorders associated with abnormal angiogenesis affecting any tissue within the body including hemangiomas and telangiectasia;
(g) proliferative disorders of bone marrow including megaloblastic disease, myelodysplastic syndromes, polycythemia vera, thrombocytosis and myelofibrosis;
(h) autoimmune disease characterized by abnormal immunological responses including multiple sclerosis, Type 1 diabetes, systemic lupus erythematosus, and biliary cirrhosis;
(i) neurodegenerative diseases and disorders characterised by degenerative changes in the structure of the neurological system including Parkinson's Disease, Alzheimer's Disease, muscular dystrophy, Lou-Gehrig Disease, and motorneurone disease;
(j) diseases and disorders associated with degenerative changes within the walls of blood vessels including atherosclerosis, stenosis, restenosis, atheroma, coronary artery disease, stroke, myocardial infarction, hypertensive vascular disease, malignant hypertension, thromboangiitis obliterans, and fibromuscular dysplasia;
(k) diseases and disorders associated with abnormal immunological responses including dermatomyositis and scleroderma;
(l) diseases and disorders associated with degenerative changes within the eye including cataracts, macular degeneration, and retinal atrophy.

9. Use of one or more compounds selected from formula (I) for the treatment, amelioration, defence against, prophylaxis and/or prevention of abnormal estrogen/androgen balance or a condition resulting from said abnormal balance in men or women.

10. Use of one or more compounds selected from formula (I) in the manufacture of a medicament for the treatment, amelioration, defence against, prophylaxis and/or prevention of abnormal estrogen/androgen balance or a condition resulting from said abnormal balance in men or women.

11. Use of one or more compounds selected from formula (I) for the manufacture of a medicament for the treatment, amelioration, defence against, prophylaxis and/or prevention of one or more diseases and disorders.

12. Use of one or more compounds selected from formula (I) in the treatment, amelioration, defence against, prophylaxis and/or prevention of one or more diseases and disorders.

13. An agent for the treatment, prophylaxis, amelioration, defence against and/or treatment of one or more diseases and disorders which comprises one or more compounds selected from formula (I) either alone or in association with one or more carriers or excipients.

14. A therapeutic composition which comprises one or more compounds selected from formula (I) in association with one or more pharmaceutical carriers and/or excipients.

15. A drink or food-stuff, which contains one or more compounds selected from formula (I).

16. One or more microorganisms which produce one or more compounds selected from formula (I).

17. A process for the production of a compound of formula (I) as defined in claim 1 according to any one or more of Schemes 1 to 8 as hereinbefore described.
